SEQUENCE LISTING (12 sequences)

SEQ ID NO: 1 - DNA, 2000 bp, Solanum lycopersicum
  source 1..2000: mol_type = genomic DNA
atgcaaataatataatatggaaggtgtgggtgtaagggtgagatggagtgtgttgaatgatggaatgaaatacaaacttacaaaatttttattattttctactttcagaaatcattttttttattttttatttttacaagaaaagccattctttattgttaaattatcttccttttttgaaaaaaaagatattgaccaatttaacattaaaattacagaaaaacacaatcatgttgcgataatagaattgcataattctgtcttaattaagtataaatcagctgactgaattctatgtggaactcaacaaatcaaccctaactttcatttcaacgtgcggtttcacaaaaccctaaaaaagttaaatcttcactttatctatcaattgacactccataacggatttagaattttaattccatgagttaagcatttctagatgtttagtattgagtcaattatatgtttgaagttataattcatgtaactttgcctatgaatttatgcttcatcagaagttatgatttcaattaaacttgtatccttccctatagatatgatatgaatttatatcatcgagttaaattacttcaagtttgacggaaatattattcttaaatttcaaacaagttgatattgattatatgaatttttaccatgaattcagaagtagaattaatatctatgtttttcttaattaaacaaaattagagcccgtttgaataggtttagtagtcggtcaaacctacttttaaatcaatttttgacttctgaaagtgttaggcaaatataaaaagtaactaaaataagttacgaagtgtctgacaaagtaaaaaatgactcaaaacaaataaaaaatgatttaaaataagtcaaaaaccaaaagtagatcccctattactttttattttttgacttaaaagtcatttcattttgattttttatttttaatttaaaagctatttttttaagccaatccagacggtctcttaatatacaggtcaaacctcattaaataaaatttaaatatttgaaagaaaagtttgagagattttaaacagcacaaggggcatattagtcaagaagaaacaaaaataacacgctttgcaataattggtgaaattttagtctgcaataaacaatcccataacatcacgtctggtttatatctggaaaaaagccatttgaatgtcattttcttggccagccatctctattatctctcttcactttaattttgagtgatactttcttcgtccatccgactcaacacacatcttttaagaaataataaattcgaagagtaattttattatatatcatcagtcacccctattggtaacacgtcatctaaatattaaaaagtaaataaaatggtaaaacatctcttgtgtttttcaaattgaataattatttttagtatagtaaacaagtaaaaatagtcgtagctagggataaagttagggtaagtagggatataatataaaaagaaagaaaagcatataagtattatgttttttcttcattgatcagtgtacaaataagaagtctttggaagttgtgtgagttttcagaaagcctttgaagttcgccggaaaatagcaatattttcaattcaagccaatcaggtctattacgttgatattttacatagcatcaaattttagaaagaaaaaaatatatgaaaaaacttaaatttcccattcttccatgcattttttaaatttttttttttttgcagattctgaaatgtttctctctgtgttcattatgacaaaattaatttgtgtttcgtgtggaactaagtcaagctttagatctatctgcaaattacataggttatagaaatatgaaagatttcatttttatatctatcaagcgcgtgcattttttttttcttttaatctttcacttatttgaaagggaagggtgcttactatctgagtaacctcctcttgtcacggaaattttggttgatcaataaaagatctccttgaaac

SEQ ID NO: 2 - DNA, 21 bp, synthetic construct
  source 1..21: mol_type = other DNA
gagcacacattgagccttgac

SEQ ID NO: 3 - DNA, 20 bp, synthetic construct
  source 1..20: mol_type = other DNA
ctttggtacatcggcattcc

SEQ ID NO: 4 - DNA, 715 bp, Solanum lycopersicum
  source 1..715: mol_type = genomic DNA
tggccacccattcgatcttttagaaagaacactctagcctctgcctcgaagaataacgaagaggttgacggaaaagctggctcaccagctctttttattaaggtcagcatggatggtgctccctatttgaggaaagtggacctcagaacctgttctgcataccaggagctatcttctgctcttgaaaaaatgttcagctgttttacaataggtcaatatggatctcatggagctcctgggaaggatatgttaagtgagagcaaattgaaggatttgcttcatggatctgagtatgtcctcacttacgaagataaggatggggactggatgcttgtcggtgatgtcccctgggagatgtttatcgatacttgcaaaaggttgaggatcatgaaaggttcagatgccattggcctggccccaagggctatggaaaagtgtcggagcagaaattagcctatctacacatgattaaagctatccatccagcatttctagtaggcgaggaagacacaaaaacgggtgaagagctcataaacttgaatcctttttaaaggcatgaacagtagctggttgttggtcttggatagctcagcatgggtagatgctttacctgattacgacatggtgttcttttatatttgcgtaatatatgtcctcttttaagtttttttttatcatgtctatttaaaaaaccgtttgatattggagtttgtct

SEQ ID NO: 5 - DNA, 24 bp, synthetic construct
  source 1..24: mol_type = other DNA
atgcaaataatataatatggaagg

SEQ ID NO: 6 - DNA, 25 bp, synthetic construct
  source 1..25: mol_type = other DNA
gtttcaaggagatcttttattgatc

SEQ ID NO: 7 - DNA, 22 bp, synthetic construct
  source 1..22: mol_type = other DNA
gagaggcttcaggaactaagca

SEQ ID NO: 8 - DNA, 22 bp, synthetic construct
  source 1..22: mol_type = other DNA
gtcatgatgaggaggaggcaat

SEQ ID NO: 9 - DNA, 23 bp, synthetic construct
  source 1..23: mol_type = other DNA
tggccacccattcgatcttttag

SEQ ID NO: 10 - DNA, 21 bp, synthetic construct
  source 1..21: mol_type = other DNA
acaaactccaatatcaaacgg

SEQ ID NO: 11 - DNA, 19 bp, synthetic construct
  source 1..19: mol_type = other DNA
ctcaggctcggtctacctg

SEQ ID NO: 12 - DNA, 22 bp, synthetic construct
  source 1..22: mol_type = other DNA
cctctgagaatccatccatagc

(19) United States
(12) Patent Application Publication
(10) Pub. No.: US 2025/0361515 A1
(43) Pub. Date: Nov. 27, 2025

(54) TISSUE-SPECIFIC PROMOTER AND USES THEREOF

(71) Applicant: UNIVERSITY OF SEOUL INDUSTRY COOPERATION FOUNDATION, Seoul (KR)

(72) Inventors: Ji-Seong KIM, Seoul (KR); Jeongeun LEE, Seoul (KR)

(73) Assignee: UNIVERSITY OF SEOUL INDUSTRY COOPERATION FOUNDATION, Seoul (KR)

(21) Appl. No.: 19/209,251

(22) Filed: May 15, 2025

(30) Foreign Application Priority Data
May 23, 2024 (KR) 10-2024-0066925

(51) Int. Cl. C12N 15/82; C07K 14/415

(52) U.S. Cl. CPC C12N 15/8229; C07K 14/415; C12N 15/8201; C12N 15/8242

(57) ABSTRACT

The present disclosure relates to a promoter that is expressed in a tissue-specific manner and uses thereof. Specifically, the promoter of the present disclosure is expressed specifically in the fruit and axillary meristem of a plant. When a fruit-producing plant is transformed with the promoter of the present disclosure, it is possible to breed a variety capable of accumulating useful products in the fruit or producing parthenocarpic fruits, without affecting other vegetative organs.

### BACKGROUND

### 1. Field of the Invention

The present disclosure relates to a tissue-specific promoter and uses thereof.

### 2. Background of the Invention

When producing transgenic plants, the 35S promoter (P35S) derived from cauliflower mosaic virus (CaMV) is mainly used to express the target gene in the plant. Since the CaMV 35S promoter drives constitutive expression of transgenes, it has been used to elucidate the functions of many plant genes, and has also been widely used to develop useful transgenic crop varieties through gene overexpression.

However, it has often been reported that although a transgene exhibits useful effects in a specific tissue, it exhibits unexpected negative effects in other tissues. Accordingly, the need for a technology that allows a gene to function only in a specific organ or tissue or at a specific time has arisen. Under this background, transformation with a transgene fused with a tissue-specific promoter has gradually attracted attention. In this case, since the desired gene is expressed only in a specific tissue, it is possible to produce a transformant that can obtain the desired phenotype while minimizing side effects. However, since the location, time, and other conditions of action are very different for each promoter, it is necessary to understand the expression conditions in detail and select the appropriate promoter required for transformation.

In the production of transgenic plants, promoters that are specifically expressed in fruits are expected to be particularly useful. If a specific gene can be regulated to be expressed only in the fruit, genes for the production of useful substances can be fused to allow the useful substances to accumulate in the fruit, and parthenocarpic fruiting, which forms fruits without fertilization and produces seedless fruits, can also be induced.

Under this background, the inventors of the present disclosure have developed a promoter that is specifically expressed in fruit tissues in plants such as tomatoes, and it is expected that the promoter of the present disclosure may be used in the breeding of new varieties that produce parthenocarpic or useful product-accumulating fruits.

### PRIOR ART DOCUMENTS

### Non-Patent Documents

(Non-Patent Document 1) Kim J S, Lee J, Ezura H. SIMBP3 Knockout/down in Tomato: Normal-Sized Fruit with Increased Dry Matter Content through Non-Liquefied Locular Tissue by Altered Cell Wall Formation. Plant and Cell Physiol. 2022 Oct. 31; 63 (10): 1485-1499.

### SUMMARY OF THE INVENTION

Accordingly, the inventors of the present disclosure have found that a promoter comprising the nucleotide sequence of SEQ ID NO: 1 specifically expresses a gene linked downstream thereof in the internal tissue of a fruit and axillary meristem.

Therefore, an object of the present disclosure is to provide a promoter that is specifically expressed in the internal tissue of the fruit of a plant or in the axillary meristem of the plant, the promoter comprising the nucleotide sequence of SEQ ID NO: 1.

Another object of the present disclosure is to provide a recombinant vector comprising the promoter.

Still another object of the present disclosure is to provide a transgenic plant transformed with the recombinant vector.

Yet another object of the present disclosure is to provide a method for producing a transgenic plant that specifically expresses a foreign gene in a fruit or axillary meristem thereof, the method comprising steps of: inserting the foreign gene into a recombinant vector comprising a promoter comprising the nucleotide sequence of SEQ ID NO: 1; and transforming a plant with the recombinant vector into which the foreign gene has been inserted.

The present disclosure relates to a promoter that is specifically expressed in the internal tissue of the fruit of a plant or in the axillary meristem of the plant, the promoter comprising the nucleotide sequence of SEQ ID NO: 1, and uses thereof.

Hereinafter, the present disclosure will be described in more detail.

One aspect of the present disclosure is a promoter that is specifically expressed in the internal tissue of the fruit of a plant or in the axillary meristem of the plant, the promoter comprising the nucleotide sequence of SEQ ID NO: 1.

The fruit refers to an organ formed by the growth of an ovary in a plant, and usually contains seeds. Generally, after a plant blooms and is fertilized, the ovary develops into a fruit. However, in the case of parthenocarpic fruits, the fruit is formed even without fertilization and does not contain seeds.

The axillary meristem is the apical meristem of the axillary bud located between the stem and the leaf, and develops into a branch by differentiation.

In addition, a variant of the promoter sequence is included within the scope of the present disclosure. The nucleotide sequence of the variant is altered, but is a nucleotide sequence having functional characteristics similar to those of the nucleotide sequence of SEQ ID NO: 1. Specifically, the promoter sequence may include a nucleotide sequence having at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 1.

The percentage of “sequence identity” to a polynucleotide may be determined by comparing two optimally aligned sequences over a comparison window, such that the portion of the polynucleotide sequence in the comparison window may comprise additions or deletions (gaps) as compared to the reference sequence (which does not comprise additions or deletions) for optimal alignment of the two sequences.

As used herein, the term “specifically” means that the expression activity of the promoter is higher in a particular tissue than in at least one other tissue within the same plant, or means that the expression level of the promoter is higher in callus or suspension-cultured cells than in other tissues within the same plant. The level of expression activity of the promoter is evaluated by comparing the expression level of the promoter in a previously measured tissue with that in another tissue using a commonly used method. Generally, the expression level of a promoter is measured by the amount of gene product (such as protein and RNA) expressed under the control of the promoter.

As used herein, the term “promoter” refers to a DNA sequence capable of controlling the expression of a coding sequence or functional RNA. The promoter consists of proximal and more distal upstream elements, and the promoter region is generally considered to be present upstream from the initiation codon, but may also exist downstream as well as in introns. The proximal elements typically include a TATA box capable of directing RNA polymerase II to initiate RNA synthesis at an appropriate transcription initiation site. The distal elements include regulatory sequences referred to as enhancers, which are additional regulatory elements that are involved in tissue-specific or time-specific expression upstream of the TATA box and located at a location where it is difficult to estimate the distance from the start codon. An enhancer is a DNA sequence capable of stimulating promoter activity and may be an innate element of the promoter or a heterologous element inserted to enhance the expression level or tissue-specificity of a promoter. Promoters may be derived in their entirety from a native gene, or be composed of different elements derived from different promoters found in nature, or even be comprised of synthetic DNA segments. It can be understood by those skilled in the art that different promoters may direct the expression of a gene in different tissues or cell types, or at different stages of development, or in response to different environmental conditions. Promoters that cause a gene to be expressed in most cell types at most times are referred to as “constitutive promoters”. New promoters of various types useful in plant cells are constantly being discovered. It is further recognized that since in most cases, the exact boundaries of regulatory sequences have not been completely defined, DNA fragments of some variation may have identical promoter activity.

Another aspect of the present disclosure is a recombinant vector comprising the promoter of the present disclosure, which is specifically expressed in the internal tissue of the fruit of a plant or in the axillary meristem of the plant.

The recombinant vector may further comprise genes involved in the production of useful products. Examples of the genes include, but are not limited to, a gene involved in lycopene accumulation, a gene involved in phenylpropanoid biosynthesis, a gene involved in GABA (γ-aminobutyric acid) accumulation, a gene involved in miraculin accumulation, etc., and any genes known in the art may be used. For example, AtMYB12, a gene involved in phenylpropanoid accumulation in tomato fruit, may be further comprised in the recombinant vector, and when the gene is transformed into a tomato, tomato fruit rich in phenylpropanoids having antioxidant and anti-inflammatory effects may be produced. As another example, GABA is a useful product that shows the effect of alleviating metabolic diseases such as diabetes and hypertension and anti-stress effects, and knocking down the SIGAD3 gene can induce GABA (γ-aminobutyric acid) in the fruit. As another example, miraculin, a glycoprotein, is a useful product that changes sourness into sweetness in the mouth, and its accumulation in the fruit can be induced by regulating the expression of the miraculin gene.

The recombinant vector may further comprise a parthenocarpy-related gene downstream of the promoter. For example, the parthenocarpy-related gene may be SlIAA9, and the recombinant vector may comprise a gene encoding a protein which inhibits the expression of the parthenocarpy-related gene SlIAA9 compared to that of the wild type or whose function has been inactivated compared to that of the wild type SlIAA9 protein, without being limited thereto, and any gene known in the art may be used. Where the recombinant vector further comprises a gene that induces parthenocarpy, when the recombinant vector is transformed into a plant, the gene that induces parthenocarpy may be expressed only in fruit tissue. As a result, the transgenic plant can produce parthenocarpic fruits, which are seedless fruits, without fertilization, and other tissues such as leaves or stems can be unaffected. Meanwhile, when a transformant is produced using the recombinant vector of the present disclosure to induce parthenocarpy, the cost required for pollination can be saved or the quality of the fruit can be improved, and parthenocarpic fruit can be formed even under extreme heat stress where fruit formation is difficult.

The term “recombinant” as used herein refers to a cell that replicates a heterologous nucleic acid, or expresses the nucleic acid, or expresses a protein encoded by a peptide, a heterologous peptide, or a heterologous nucleic acid. The recombinant cell may express a gene or a fragment of the gene which is not found in the native form of said cell, either in sense or antisense form. Further, the recombinant cell may express a gene found in the native form of the cell, but the gene is a modified gene reintroduced into the cell by an artificial means.

The term “vector” as used herein means a means for expressing a target gene in a host cell. Examples of the vector include plasmid vectors, cosmid vectors, and viral vectors such as a bacteriophage vector, an adenovirus vector, a retrovirus vector, and an adeno-associated virus vector.

The vector that may be used as the recombinant vector may be constructed by manipulating a plasmid (e.g., pSC101, pGV1106, pACYC177, ColE1, pKT230, pME290, pBR322, pUC8/9, pUC6, pBD9, pHC79, pIJ61, pLAFR1, pHV14, pRadgro, pKM212 series, pGEX series, pET series, pUC19, etc.) frequently used in the art, a phage (e.g., λgt4λB, λ-Charon, λΔz1, M13, etc.) or a virus (e.g., SV40, etc.), and for example, may be constructed by manipulating a pBI-sense, antisense GW vector, without being limited thereto.

The vector may typically be constructed as a vector for cloning or a vector for expression. The vector for expression may be a conventional one that is used in the art to express foreign protein in a plant, animal or microorganism, and may be constructed through various methods known in the art.

The recombinant vector may be constructed for use in a prokaryotic or eukaryotic host cell. For example, when the vector used is an expression vector and a prokaryotic cell is used as a host cell, the vector usually comprises a strong promoter capable of initiating transcription (e.g., pLλ promoter, CMV promoter, trp promoter, lac promoter, tac promoter, T7 promoter, etc.), a ribosome binding site for initiating translation, and a transcription/translation termination sequence. When a eukaryotic cell is used as a host cell, the vector may comprise an origin of replication acting in the eukaryotic cell. The origin of replication may be, for example, f1 origin of replication, SV40 origin of replication, pMB1 origin of replication, adeno origin of replication, AAV origin of replication, or BBV origin of replication, without being limited thereto. In addition, the vector may comprise a promoter derived from the genome of mammalian cells (e.g., a metallothionein promoter) or a promoter derived from mammalian viruses (e.g., an adenovirus late promoter, a vaccinia virus 7.5K promoter, a SV40 promoter, a cytomegalovirus promoter, and a tk promoter of HSV), and generally has a polyadenylation sequence as a transcription termination sequence.

A transformant may be produced by inserting the recombinant vector into a host cell, and the transformant may be obtained by introducing the recombinant vector into an appropriate host cell. Any host cell known in the art may be used as long as it is capable of stably and continuously cloning or expressing the expression vector.

When a eukaryotic cell is transformed with the vector to produce a recombinant microorganism, yeast (Saccharomyces cerevisiae), insect cells, plant cells, or animal cells, such as Sp2/0, CHO (Chinese hamster ovary) K1, CHO DG44, PER.C6, W138, BHK, COS7, 293, HepG2, Huh7, 3T3, RIN, and MDCK cell lines, may be used as host cells, without being limited thereto.

In order to transfer (introduce) a polynucleotide or a recombinant vector containing the same into a host cell, a transfer method widely known in the art may be used. For example, when the host cell is a prokaryotic cell, a method such as conjugation, the CaCl₂ method, or electroporation may be preferably used, and when the host cell is a eukaryotic cell, a method such as Agrobacterium-mediated transformation, microinjection, calcium phosphate precipitation, electroporation, liposome-mediated transfection or gene bombardment may be preferably used. In the present disclosure where the host cell is a plant cell, a more preferable transformation method may be Agrobacterium-mediated transformation, without being limited thereto.

The recombinant expression vector of the present disclosure may be used as a transient expression vector that can transiently express a foreign gene in a plant into which the foreign gene has been introduced, or as a plant expression vector that can permanently express a foreign gene in a plant into which the foreign gene has been introduced.

Another aspect of the present disclosure is a transgenic plant transformed with the recombinant vector. The transgenic plant can express a foreign gene only in the fruit or axillary meristem to produce a useful product, such as a parthenocarpic fruit or a fruit in which a useful product is accumulated.

The “plant cell” used in plant transformation may be any plant cell. “Plant tissue” includes differentiated or undifferentiated plant tissues, for example, but not limited to, fruit, stem, leaf, pollen, seed, cancerous tissue, and various types of cells that are used for culture, i.e., single cell, protoplast, shoot, and callus tissue. The plant tissue may be in planta, or in organ culture, tissue culture, or cell culture.

Another aspect of the present disclosure is a method for producing a transgenic plant that specifically expresses a foreign gene in a fruit or axillary meristem thereof, the method comprising steps of: inserting a foreign gene into a recombinant plant expression vector comprising the promoter comprising the nucleotide sequence of SEQ ID NO: 1; and transforming a plant with the recombinant plant expression vector into which the foreign gene has been inserted.

The foreign gene may be any gene that is desired to be expressed in a plant, and may be positioned downstream of the promoter in a recombinant plant expression vector comprising the promoter of the present disclosure, which is specifically expressed in the fruit or axillary meristem, and the foreign gene may also be expressed in fusion with a reporter gene as needed. The method for transforming a plant with a recombinant plant expression vector comprising a promoter that is specifically expressed in the fruit or axillary meristem of the recombinant plant may be performed as described above.

In a method according to one embodiment of the present disclosure, the plant may be a food crop selected from the group consisting of rice, wheat, barley, corn, soybean, potato, wheat, red bean, oat, and sorghum; a vegetable crop selected from the group consisting of Arabidopsis, Chinese cabbage, radish, pepper, strawberry, tomato, watermelon, cucumber, cabbage, melon, pumpkin, green onion, onion, and carrot; a special crop selected from the group consisting of ginseng, tobacco, cotton, sesame, sugarcane, sugar beet, perilla, peanut, and rapeseed; a fruit tree selected from the group consisting of apple trees, pear trees, jujube trees, peaches, kiwis, grapes, citrus fruits, persimmons, plums, apricots, and bananas; a flowering plant selected from the group consisting of roses, gladiolus, gerberas, carnations, chrysanthemums, lilies, and tulips; or a forage crop selected from the group consisting of ryegrass, red clover, orchardgrass, alfalfa, tall fescue, and perennial ryegrass. Preferably, the plant may be a dicotyledonous plant such as tomato, Arabidopsis, potato, eggplant, tobacco, pepper, burdock, crown daisy, lettuce, bellflower, spinach, chard, sweet potato, celery, carrot, water parsley, parsley, Chinese cabbage, cabbage, mustard greens, watermelon, melon, cucumber, pumpkin, gourd, strawberry, soybean, mung bean, kidney bean, or pea. More preferably, the plant is tomato.

The present disclosure relates to a tissue-specific promoter and uses thereof, and specifically, the promoter of the present disclosure, which comprises the nucleotide sequence of SEQ ID NO: 1, is specifically expressed only in the internal tissue of a fruit and in axillary meristem.

When the promoter of the present disclosure is fused with a foreign gene and transformed into a plant, it is specifically expressed only in the internal tissue of a fruit and in axillary meristem, and thus it is possible to breed a new variety that can produce fruits with useful products accumulated therein or parthenocarpic fruits without any particular effect on the vegetative organs.

### BRIEF DESCRIPTION OF THE DRAWINGS

FIG. 1 shows the expression level of SIMBP3 (Solyc06g064840) mRNA on various tissues of the wild type (WT) (DAA=days after anthesis).

FIG. 2 schematically shows the T-DNA region of the vectors for generation of transgenic tomato plants. P35S-GUS denotes CaMV 35S promoter-GUS line, P19-GUS denotes SIMBP3 promoter-GUS line, P35S-SlIAA9i denotes CaMV 35S promoter-SlIAA9 RNA interference line, and P19S-SlIAA9i denotes SIMBP3 promoter-SlIAA9 RNA interference line.

FIG. 3 shows the results of GUS staining in transgenic tomato plant (solid bar=1 cm, dotted bar=1 mm).

FIG. 4 shows the results of measuring the expression level of SIMBP3 (Solyc06g064840) in the various tissues of 8 DAA (days after anthesis) fruit from wild type (WT).

FIG. 5 relates to promoter activity in 30-day-old stem. Specifically, (A) shows magnifying observation of GUS staining (bar=1 mm), (B) shows the expression level of SIMBP3 mRNA in axillary bud from wild type (WT).

FIG. 6 relates to the vegetative phenotype and SlIAA9 mRNA level of SIMBP3 promoter-SlIAA9 RNA interference lines. Specifically, (A) shows the form of 35-day-old plants (bar=5 cm), (B) shows the shape of leaves (bar=1 cm), (C) shows plant heights, (D) shows the number of branches, and (E) shows the expression levels of SlIAA9 mRNA in 5-cm length leaves. Wild type (WT), SIMBP3 promoter-SlIAA9 RNA interference lines (MG102, 103, 106, 203 and 210), and CaMV 35S promoter-SlIAA9 RNA interference line (P35S-SlIAA9i).

FIG. 7 relates to parthenocarpic fruit formation and SlIAA9 mRNA expression level in fruits. Specifically, (A) shows mature fruit and cross-section of the fruit (bar=1 cm), (B) shows the expression levels of SlIAA9 mRNA in locular tissue of green fruit, (C) shows the fresh weight of fruit (n=25), (D) shows the formation rate of parthenocarpic fruit (n=60), and (E) shows the percentage of fruit set after pollination (n=30).

FIG. 8 shows time-dependent changes in fruit size in the wild type, P19-SlIAA91 line, and P35S-SlIAA91 line.

FIG. 9 relates to tomato fruit formation under heat stress. Specifically, (A) shows plant growth (bar=4 cm), (B) shows parthenocarpic fruit formation (bar=1 cm), and (C) shows the fresh weight of fruit (n=20). Wild type (WT), SIMBP3 promoter-SlIAA9 RNA interference lines (MG102, 103, 106, 203 and 210), and CaMV 35S promoter-SlIAA9 RNA interference line (P35S-SlIAA91).

FIG. 10 relates to extreme heat stress and tomato fruit formation. Specifically, (A) shows plant growth (bar=4 cm), (B) shows parthenocarpic fruit formation (bar=1 cm), and (C) shows the fresh weight of fruit (n=7). Wild type pollinated (WT pol), SIMBP3 promoter-SlIAA9 RNA interference lines (MG106 and 203) and CaMV 35S promoter-SlIAA9 RNA interference line (P35S-SlIAA9i).

### DETAILED DESCRIPTION OF THE INVENTION

Hereinafter, one or more embodiments will be described in more detail by way of examples. However, these examples are intended to illustrate one or more embodiments, and the scope of the present disclosure is not limited to these examples.

### Example 1. Plant Material and Growth Conditions

Micro-Tom, a dwarf cultivar of tomato, was used in the present disclosure. Seeds were placed on filter paper together with distilled water for 5 days at 25° C. Then, the seedlings were transplanted onto rockwool cube (MM40/40; Grodan, Roermond, The Netherlands), irrigated with a nutrient solution (Coseal, Kunsan, Korea) with an electrical conductivity (EC) of 2.2 dS m⁻¹, and incubated under 16-hr light/8-hr dark light cycles at a light intensity of 150 μmol m⁻² s⁻¹ and a temperature of 25° C.

For the preparation of fruits, floral bud was emasculated one day before anthesis or artificially pollinated by vibration at anthesis. Six fruits per plant were maintained for the experiment.

For the heat stress experiment, the 30-day old plants were cultivated in a plant growth chamber (VISION SCIENTIFIC, Daejeon, Korea) with the cycles of 16-hr light at 34° C. and 8-hr dark at 26° C., under 60% humidity and 150 μmol m⁻² s⁻¹ light intensity for one-month. For fruit formation of wild type (WT) under heat stress, artificial pollination was carried out for all opened flowers every day.

### Example 2. DNA and RNA Extraction and cDNA Synthesis

Genomic DNA and total RNA were isolated using a DNeasy plant mini kit (Qiagen) and a TakaRa MiniBEST Universal RNA Extraction Kit (Takara) according to the manufacturers' instructions, respectively. First-strand CDNA was synthesized from 1 μg of total RNA using the PrimeScript II 1^{st} strand CDNA Synthesis Kit (Takara) and oligo dT primers. The synthesized cDNA was subjected to PCR to verify genomic DNA-free cDNA with a set of DNAJ gene primers (forward, GAGCACACATTGAGCCTTGAC (SEQ ID NO: 2); reverse, CTTTGGTACATCGGCATTCC (SEQ ID NO: 3)).

### Example 3. Vector Construction and Tomato

### Transformation

The promoter fragments of SIMBP3 (Solyc06g064840) were amplified from the genomic DNA and a 715-bp SlIAA9 (Solyc04g076850) amplicon (SEQ ID NO: 4) was amplified from ovary cDNA with 1 U of KOD Plus Neo (Toyobo), 1× buffer, 0.2 mM dNTPs, 1.5 mM MgSO₄, and 0.2 μM primers.

The thermal cycling procedure was as follows, and (ii) to (iv) were repeated for 28 cycles: (i) pre-denaturation for 5 min at 98° C., (ii) denaturation for 30 sec at 98° C., (iii) annealing for 30 sec at 60° C., (iv) extension for 1 min at 68° C., and (v) final extension at 68° C. for 3 min.

DNA fragments containing a 2000-bp promoter region were replaced between HindIII (5′) and XbaI (3′) restriction sites of pBI121 vector or between AvrII (5′) and XhoI (3′) restriction sites of the pBI-sense, antisense GW vector (Inplanta Innovations) using an In-Fusion cloning method (Takara). Subsequently, 715-bp SlIAA9 fragments were cloned into the pCR8/GW/TOPO vector and inserted into the pBI-sense, antisense GW vector using Gateway LR Clonase II Enzyme mix (Thermo Fisher). The primer sequences for vector construct are shown in Table 1 below.

TABLE 1

| | | | SEQ ID | |
|---|---|---|---|---|
| Locus | Definition | Objective | NO | Sequence (5′-3′) |
| Solyc06g064840 | SlMBP3 | promoter | SEQ ID | Forward, |
| | | region in | NO: 4 | ATGCAAATAATATAATATGGAAGG |
| | | vector | | Reverse, |
| | | | | GTTTCAAGGAGATCTTTTATTGATC |
| | | qRT-PCR | SEQ ID | Forward, GAGAGGCTTCAGGAACTAAGCA |
| | | | NO: 5 | Reverse, GTCATGATGAGGAGGAGGCAAT |
| Solyc04g076850 | SlIAA9 | gene in | SEQ ID | Forward, TGGCCACCCATTCGATCTTTTAG |
| | | vector | NO: 6 | Reverse, ACAAACTCCAATATCAAACGG |
| | | qRT-PCR | SEQ ID | Forward, CTCAGGCTCGGTCTACCTG |
| | | | NO: 7 | Reverse, CCTCTGAGAATCCATCCATAGC |

Transgenic tomato was obtained by Agrobacterium-mediated transformation. The transgenic lines were subjected to PCR using promoter and gene-specific primer sets and Southern blot analysis to select the homozygous-independent lines.

### Example 4. Quantitative Reverse Transcription PCR (qRT-PCR)

The expression levels of the genes were assessed using the combination of Mic qPCR Cycler system (Bio Molecular Systems, Queensland, Australia) and TB Green Premix Ex Taq (Tli RNaseH Plus) following the manufacturer's protocol. The PCR mixture was composed of cDNA template, 1×TB Green Premix Ex Taq and 0.2 μM primer set for qRT PCR. Dissociation curve analysis was also carried out to confirm primer compatibility. The expression level of the target gene was calculated by the standard curve method with three biological replicates and SAND was used as a reference gene. The primer set used for qRT-PCR is described in Table 1 above.

### Example 5. GUS Staining Assay

Histochemical β-glucuronidase (GUS) analysis was performed using a slight modification of the 5-bromo-4-chloro-3-indolyl-b-D-glucuronide (X-Gluc) solution previously described (Jefferson, R. (1987). The solution was composed of 50 mM phosphate buffer (pH 8.0), 0.1% triton X-100, 0.5 mM potassium hexacyanoferrate (II), 0.5 mM potassium hexacyanoferrate (III), 1 mM X-Gluc and 20% methanol (Sigma Aldrich, St. Louis, USA). The tissue was immersed in X-Gluc solution and vacuum-filtrated for 1 hour and treated at 37° C. for 16 hours, followed by washing with 70% ethanol several times.

### Experimental Results

### (1) Ovary- and Fruit-Specific Expression of SIMBP3

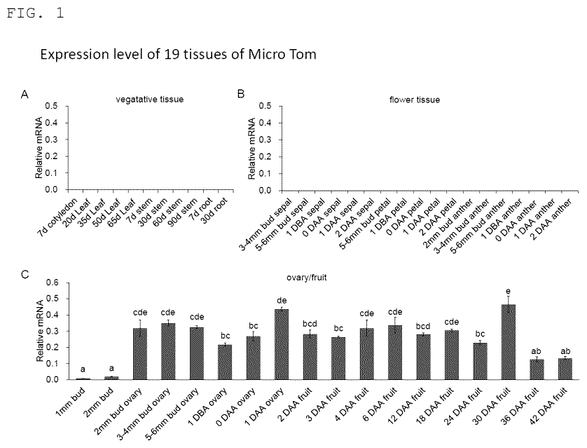

To determine the expression pattern of SIMBP3, mRNA levels in vegetative tissues (leaves, stems, and roots), flower tissues (sepals, petals, and anthers), and ovary/fruit were examined. It was confirmed that the SIMBP3 was not expressed in all tested vegetative tissues (young and old leaves, stems, and roots) but also in flower tissues (sepals, petals and anthers in various developmental stage) except ovaries to develop into fruits (FIG. 1). This gene was consistently expressed from the early development stage of ovary to mature fruit. Namely, SIMBP3 was not expressed in vegetative tissues like leaf, stem, root, and flower tissues over the whole developmental course except ovary/fruit, suggesting that SIMBP3 is a feasible approach to develop ovary/fruit tissue-specific promoter.

### (2) β-Glucuronidase (GUS) Analysis of the Promoter Activity of SIMBP3

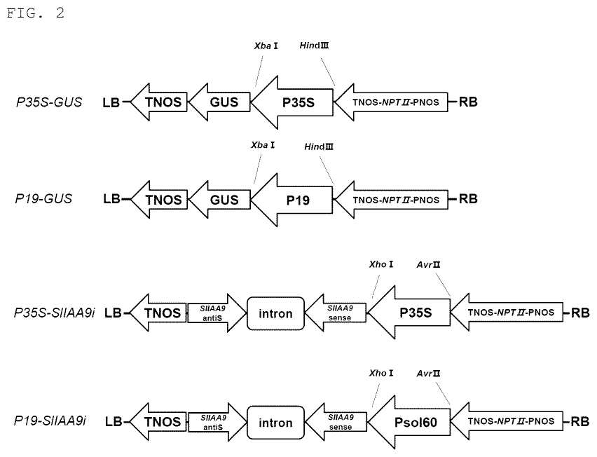

To confirm the promoter activity through GUS expression, pBI121 was used as a backbone vector for tomato transformation. The 2,000-bp region (SEQ ID NO: 1) upstream from the start codon of SIMBP3 was inserted into pBI121 vector instead of Cauliflower Mosaic Virus (CaMV) 35S promoter (P35S) region (FIG. 2). Intact pBI121 vector was used as a control for P35S.

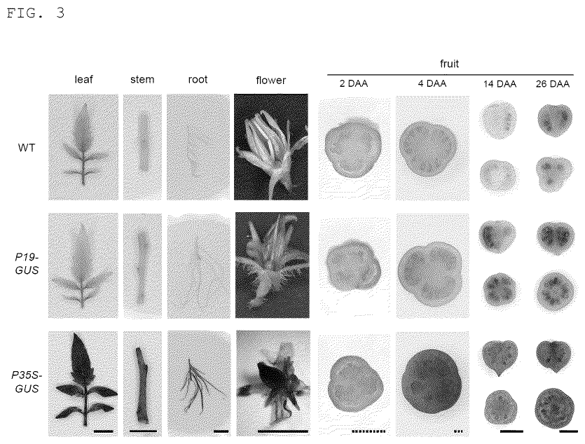

GUS histochemical analysis was carried out with more than three independent T₂ lines for each promoter lines and they showed identical GUS staining pattern. While no assayed tissues from wild type (WT) exhibited blue, all tissues from P35S-GUS lines showed blue color after GUS staining, indicating that P35S induced GUS expression in all tissues (FIG. 3). In P19-GUS lines, the GUS staining was detected as blue in seed coats, placenta and locular tissues and pale blue in septa and pericarp of fruit, indicating that the promoter of SIMBP3 confers expression gene in the corresponding tissues.

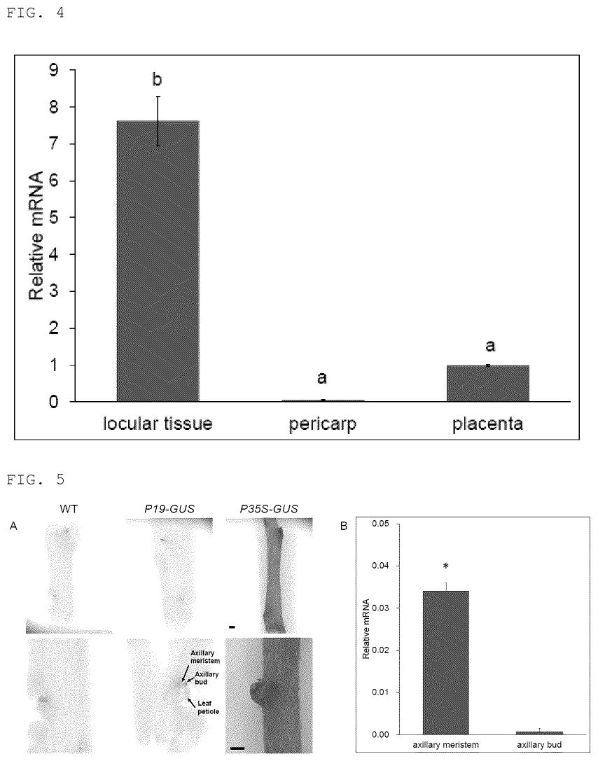

For further confirmation, the present inventors measured the mRNA expression level of SIMBP3 in the fruit at 8 DAA (days after anthesis), although our previous study showed that SIMBP3 was also expressed in placenta and septa of mature green fruit at 30 DAA. Consistent with where the GUS was detected, the SIMBP3 was expressed in lower level in placenta but rarely expressed in pericarp compared with that in locular tissues (FIG. 4).

Meanwhile, blue stain was observed near axillary buds of stem (FIG. 5). As the result of detailed observation with an optical microscope, GUS staining was observed in the axillary meristem (FIG. 5A). Similar to the result of GUS staining, the expression of SIMBP3 was slightly found in axillary meristem and rarely detected in axillary bud. These results suggested that the promoter of SIMBP3 conferred gene expression predominantly in the locular tissues of fruit and seed coat, and slightly in the placenta of fruit and axillary meristem of stem.

### (3) Vegetative Phenotype of Transgenic Tomato Plant

For a gene to function properly, the location and timing of gene expression are important. Usually, fruit develops from ovary, and after successful fertilization, fruit formation begins while the ovule, which is a source of hormones for cell division and expansion, develops into a seed.

However, ubiquitous knockout/down of SlIAA9, a negative regulator of auxin response, induces the initiation of fruit formation even before fertilization. However, it is known that ubiquitous knockout/down of SlIAA9 also induces detrimental effect like fused leaves causing reduction of leaf area for photosynthesis.

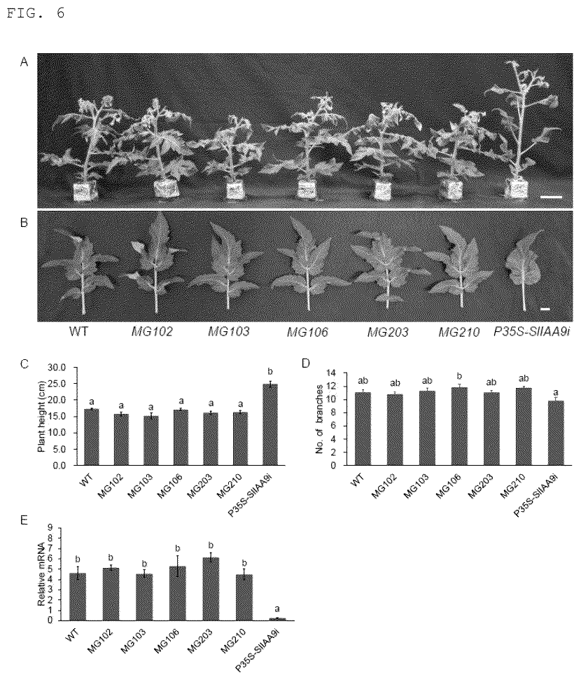

The SlIAA9 mRNA was gradually increased and highly accumulated in integument of ovule, placenta, and funiculus from when the fertilization occurred. In addition, ovule- and placenta-specific downregulation of SlIAA9 induced parthenocarpic fruit formation. These suggest that the knockdown of SlIAA9 in ovule and placenta is sufficient for fruit formation. As can be seen in FIGS. 1C and 3, it was confirmed that the SIMBP3 promoter may be a proper candidate for fruit setting through tissue-specific SlIAA9 downregulation (knockdown) without additional effect on vegetative tissues. To generate parthenocarpic tomato plants without an effect on vegetative tissue growth, the fusion construct of SIMBP3 promoter and SlIAA9 interference was transformed into tomato plant (P19-SlIAA9i line; FIG. 2). CaMV 35S promoter-SlIAA9 RNA interference lines (P35S-SlIAA91 lines)) showed an abnormal plant form such as increased plant height and fused leaves (FIGS. 2 and 6). On the other hand, P19-SlIAA91 lines (MG102, 103, 106, 203 and 210) presented leaves and plant height similar to those of wild-type (WT) tomato (FIG. 6).

Meanwhile, because the SIMBP3 promoter also induced downstream GUS gene expression in axillary meristem involved in growth of axillary bud into branch (FIG. 5), the effect of SlIAA9 downregulation in axillary meristem was examined by observation of branch development. While the P19-SlIAA91 lines formed similar numbers of branches to those of wild-type (WT), the P35S-SlIAA91 lines produced fewer branches compared to those of WT (FIG. 6D).

To further confirm that the SlIAA9 expression was not affected by the SIMBP3 promoter in the vegetative tissue in P19-SlIAA9i lines, the mRNA expression level of SlIAA9 was examined in leaves. The P19-SlIAA91 lines exhibited similar SlIAA9 mRNA expression levels to those of the WT had a leaf shape similar to that of the WT, whereas P35S-SlIAA9i lines showed downregulated SlIAA9 expression (FIG. 6E). These results suggest that SlIAA9 knockdown controlled by SIMBP3 promoter does not induce pleiotropic effect on vegetative tissues. This means that the SIMBP3 promoter specifically induces downstream gene expression only in the internal tissue of the fruit and the axillary meristem.

### (4) Evaluation of Parthenocarpic Fruit Formation of Transgenic Tomato Plant

The formation of parthenocarpic fruit was evaluated after emasculation under 25° C. temperature condition. The ovary was considered as having become a fruit when it grew to a fresh weight of 0.5 g or more and changed color from green to orange or red.

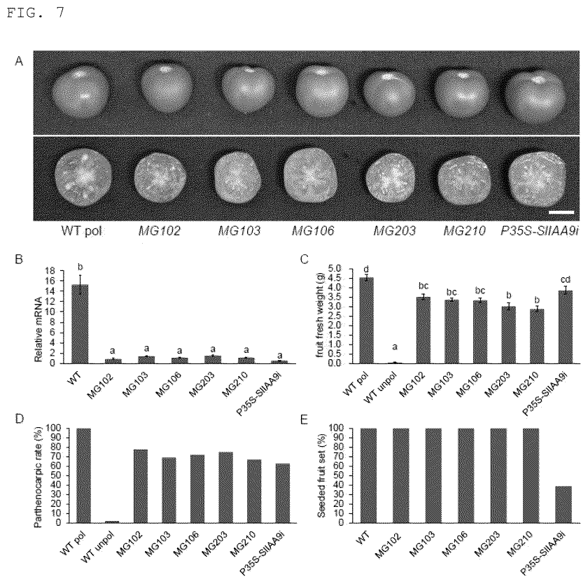

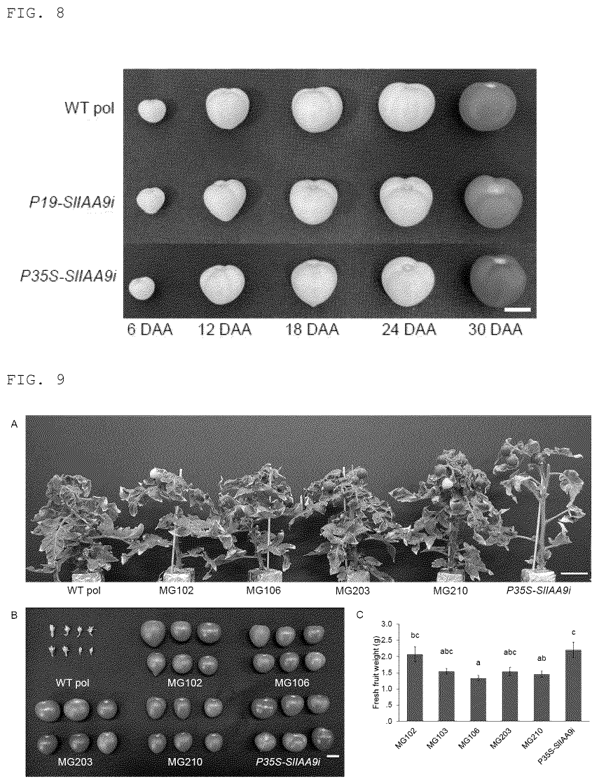

It was confirmed that P19-SlIAA91 and P35S-SlIAA91 lines formed parthenocarpic fruits (FIG. 7A). To explain parthenocapic fruit formation in P19-SlIAA91 and P35S-SlIAA9i lines, the mRNA level of SlIAA9 was measured. As the result, the mRNA expression level was downregulated in P19-SlIAA91 and P35S-SlIAA9i lines as expected, indicating that the knocked-down expression of SlIAA9 mRNA was responsible for parthenocarpic fruit formation in P19-SlIAA91 and P35S-SlIAA9i lines (FIG. 7B).

The fresh weight of parthenocarpic fruit of P19-SlIAA9i lines was slightly lighter than that of P35S-SlIAA91 lines as well as that of pollinated fruit of the wild type (WT) (FIG. 7C).

While unpollinated wild type (WT) exhibited a fruit formation rate of 5.4%, the P19-SlIAA91 and P35S-SlIAA91 lines showed parthenocarpic fruit formation abilities of up to 78% and 63%, respectively (FIG. 7D).

Meanwhile, the rate of fruit formation by pollination was also examined. As a result, merely about 40% of the pollinated ovaries in P35S-SlIAA9i lines formed normal seeded fruits, whereas 100% of those in P19-SlIAA91 lines developed into normal seeded fruits, similar to WT (FIG. 7E). These results suggest that P19-SlIAA9i lines have facultative that it is parthenocarpic fruit formation, indicating possible to choose whether or not the fruit will be parthenocarpic.

### (5) Fruit Formation Under Heat Stress Conditions

Increased temperature compared to the appropriate temperature during the day reduces pollen release and viability, thereby reducing fruit set. In addition, this reduction in fruit set is induced even upon short-term exposure to increased temperature.

To evaluate the parthenocarpic fruit setting ability of P19-SlIAA9i lines under heat stress conditions, the plants were cultivated for one month at 34° C. Pollinated wild-type (WT) and unpollinated P35S-SlIAA91 lines were used as control.

For the heat stress experiment, the 30-day-old plants were cultivated in the growth chamber (VISION SCIENTIFIC, Daejeon, Korea) with the cycles of 16-hr light at 34° C. and 8-hr dark at 26°, under 60% humidity and 150 μmol m⁻² s⁻¹ light intensity for one month. For the fruit formation of wild type (WT) under heat stress, artificial pollination was carried out for all opened flowers every day.

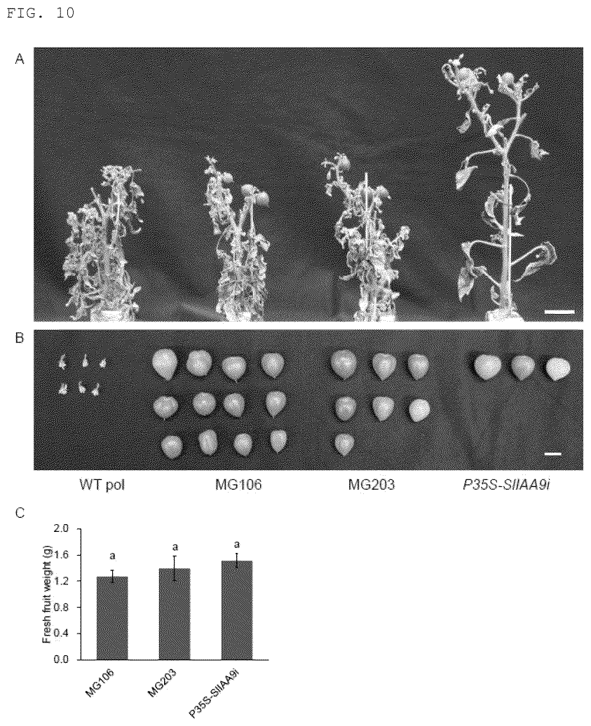

In the case of vegetative organs, all tested lines showed similar phenotypes without severe heat-induced damage compared to the lines cultivated at the appropriate temperature of 25° C., which is consistent with previously reported results (FIG. 9A).

In fruit setting, pollinated WT was totally failed to produce fruits, whereas P19-SlIAA91 lines and P35S-SlIAA91 lines formed parthenocarpic fruits (FIG. 9B). Meanwhile, it is known that the weight of fruits decreases under heat stress, and it was confirmed that the fresh weight of fruits of P19-SlIAA91 lines and P35S-SlIAA91 lines decreased slightly compared to those at the appropriate temperature (FIG. 9C).

Additionally, the present inventors examined the parthenocarpic ability of P19-SlIAA9i lines and P35S-SlIAA91 lines under extreme heat stress. At a daytime temperature of 36° C., vegetative tissues were severely damaged and wilted (FIG. 10A). However, it was confirmed that, before the plants completely wilted, P19-SlIAA9i lines and P35S-SlIAA91 lines began to bear fruit and formed parthenocarpic fruits, and the weight of the fruit was lighter than that of fruit at the appropriate temperature or a slightly higher temperature (FIG. 10B and FIG. 10C).

So far, the present disclosure has been described with reference to the preferred embodiments. Those of ordinary skill in the art to which the present disclosure pertains will appreciate that the present disclosure may be embodied in modified s without departing from the essential characteristics of the present disclosure. Therefore, the disclosed embodiments should be considered from an illustrative point of view, not from a restrictive point of view. The scope of the present disclosure is defined by the claims rather than the foregoing description, and all differences within the scope equivalent thereto should be construed as being included in the present disclosure.

What is claimed is:

1. A promoter that is specifically expressed in an internal tissue of a fruit of a plant or in an axillary meristem of the plant, the promoter comprising the nucleotide sequence of SEQ ID NO: 1.

2. A recombinant vector comprising the promoter of claim 1.

3. The recombinant vector of claim 2, further comprising, downstream of the promoter, a gene involved in production of useful products.

4. The recombinant vector of claim 2, further comprising, downstream of the promoter, a gene encoding a protein which inhibits expression of SlIAA9, a parthenocarpy-related gene, compared to that of wild type or whose function has been inactivated compared to that of a wild type SlIAA9 protein.

5. A method for producing a transgenic plant that specifically expresses a foreign gene in a fruit or axillary meristem thereof, the method comprising steps of:
inserting the foreign gene into a recombinant vector comprising a promoter comprising the nucleotide sequence of SEQ ID NO: 1; and
transforming a plant with the recombinant vector into which the foreign gene has been inserted.

6. The method of claim 5, wherein the foreign gene is a gene involved in production of useful products.

7. The method of claim 5, wherein the foreign gene is a gene encoding a protein which inhibits expression of SlIAA9, a parthenocarpy-related gene, compared to that of wild type or whose function has been inactivated compared to that of a wild type SlIAA9 protein.
